(19) United States
(12) Patent Application Publication
(10) Pub. No.: US 2023/0227804 A1
(43) Pub. Date: Jul. 20, 2023

(54) Nucleic Acids, Vectors, Host Cells and Methods for Production of Beta-Fructofuranosidase from Aspergillus Niger

(71) Applicant: Revelations Biotech Pvt Ltd, Hyderabad, Telangana (IN)

(72) Inventors: Ravi Chandra Beeram, Hyderabad, Telangana (IN); Dipanwita Sinha, Hyderabad, Telangana (IN); Bharath Babu Musuku, Secunderabad, Telangana (IN); Chiranjeevi Are, Hyderabad, Telangana (IN); Deepika Kumar, Hyderabad, Telangana (IN)

(21) Appl. No.: 17/779,016

(22) PCT Filed: Nov. 27, 2020
(86) PCT No.: PCT/IN2020/050985

§ 371 Date: May 23, 2022

(30) Foreign Application Priority Data
Nov. 27, 2019 (IN) 201941048686

(51) Int. Cl. C12N 9/26; C12N 15/81

(52) U.S. Cl. CPC C12N 9/2431; C12Y 302/01026; C12N 15/815

(57) ABSTRACT

The present invention provides nucleic acids, vectors, host cells and methods for production of beta-fructofuranosidase from Aspergillus niger. The invention represents an advancement in the field of genetic engineering and provides methods for obtaining high yield of a novel recombinant β-fructofuranosidase encoded by fopA gene of Aspergillus niger as a secreted protein.

### FIELD OF INVENTION

The present invention relates to the field of genetic engineering. More specifically, the invention is directed towards obtaining improved production of a novel recombinant β-fructofuranosidase, encoded by fopA gene of Aspergillus niger as a secreted protein.

### BACKGROUND

Fructose oligomers, also known as fructooligosaccharides (FOS) constitute a series of homologous oligosaccharides. Fructooligosaccharides are usually represented by the formula GFₙ and are mainly composed of 1-kestose (GF2), nystose (GF3) and β-fructofuranosylnystose (GF4), in which two, three, and four fructosyl units are bound at the β-2,1 position of glucose.

Fructooligosaccharides (FOS) are characterized by many beneficial properties such as low sweetness intensity and usefulness as a prebiotic. Due to the low sweetness intensity (about one-third to two-third as compared to sucrose) and low calorific values (approximately 0-3 kcal/g), fructooligosaccharides can be used in various kinds of food as a sugar substitute. Further, as a prebiotic, fructooligosaccharides have been reported for being used as protective agents against colon cancer, enhancing various parameters of the immune system, improving mineral adsorption, beneficial effects on serum lipid and cholesterol concentrations and exerting glycemic control for controlling obesity and diabetes (Dominguez, Ana Luisa, et al. “An overview of the recent developments on fructooligosaccharide production and applications.” Food and bioprocess technology 7.2 (2014): 324-337.)

However, fructooligosaccharides are found only in trace amounts as natural components in fruits, vegetables, and honey. Due to such low concentration, it is practically impossible to extract fructooligosaccharides from food.

Attempts have been made to produce fructooligosaccharides through enzymatic synthesis from sucrose by microbial enzymes with transfructosylation activity. However, the major constraints in the previous attempts have been the lower catalytic efficiency, feedback inhibition of the enzyme by glucose leading lower FOS yields and the requirement of longer time periods for conversion of sucrose by the enzymes expressed in the recombinant host system. Further, industrial production of microbial enzymes exhibiting transfructosylation activity is challenging due to additional limitations associated with large scale expression of enzyme, enzyme stability, fermentation and purification processes.

Commercial-scale production of fructooligosaccharides requires identification and mass production of efficient enzymes. Due to the aforesaid limitations, the production of microbial enzymes with efficient transfructosylation activity is a costly affair which in-turn increases the production cost of fructooligosaccharides.

Thus, there is a long-felt need for identifying and providing efficient, cheap and industrially scalable means for the production of microbial enzymes with superior transfructosylation activity, which in turn lowers the cost of production of fructooligosaccharides.

### SUMMARY OF THE INVENTION

### Technical Problem

The technical problem to be solved in this invention is to identify and improve the yield of a novel β-fructofuranosidase (UniProtKB: Q96VC5_ASPNG) of Aspergillus niger.

### The Solution to the Problem

The problem has been solved by overexpression of a novel β-fructofuranosidase of Aspergillus niger by engineering nucleic acid sequences, protein sequences, promoters, recombinant vectors, host cells and secretory signal peptides for achieving high yield of novel recombinant β-fructofuranosidase.

Additionally, the fermentation strategy has been modified to obtain a high yield of about 2-5 gm/L recombinant β-fructofuranosidase.

### Overview of the Invention

The present invention relates to nucleic acids, protein sequences, vectors and host cells for recombinant expression of a novel β-fructofuranosidase. The present invention also relates to precursor peptides containing signal peptides fused to a novel β-fructofuranosidase enzymes which enable generation of higher yield of the efficient enzyme as a secretory protein.

The invention also relates to a process for the expression of a novel recombinant β-fructofuranosidase as a secreted protein. The β-fructofuranosidase concentration is found to be about 2-5 gm/L. The enzyme exhibits almost 85% purity after filtration, which eliminates the need for costly chromatographic procedures.

### BRIEF DESCRIPTION OF DRAWINGS

The features of the present disclosure will become fully apparent from the following description taken in conjunction with the accompanying figures. With the understanding that the figures depict only several embodiments in accordance with the disclosure and are not to be considered limiting of its scope, the disclosure will be described further through the use of the accompanying figures.

FIG. 1 depicts the sequence alignment of the native fopA gene and the modified fopA gene encoding β-fructofuranosidase.

FIG. 2 represents the construction scheme of pPICZαA vector.

FIG. 3 depicts the results of the restriction digestion analysis performed on the recombinant plasmid pPICZαA-fopA.

FIG. 4 depicts the results of the colony PCR screening performed on the Pichia integrants.

FIG. 5 depicts the expression of β-fructofuranosidase upon induction from the recombinant Pichia pastoris host cells.

FIG. 6 (a) depicts the SDS-PAGE analysis of samples collected at different time intervals during fermentation of Pichia pastoris KM71H strain expressing recombinant β-fructofuranosidase enzyme. FIG. 6 (b) depicts the SDS-PAGE analysis of recombinant β-fructofuranosidase enzyme after purification.

FIG. 7 depicts the Glucose standard curve used for the estimation of the activity of β-fructofuranosidase enzyme.

FIG. 8 depicts the generation of fructooligosaccharides (FOS) from sucrose and recombinant β-fructofuranosidase enzyme.

FIG. 9 depicts the HPLC analysis chromatogram of FOS samples.

### BRIEF DESCRIPTION OF SEQUENCES AND SEQUENCE LISTING

SEQ ID NO: 1—Amino acid sequence of novel β-fructofuranosidase (654 amino acids)

SEQ ID NO: 2—Modified nucleic acid sequence of the gene encoding novel β-fructofuranosidase (1965 base pairs)

TABLE 1
Modified Signals Peptides used

| Sr. | Modified Signal | SEQ ID | | Length |
|---|---|---|---|---|
| No. | Peptide (Source) | NO | Amino Acid Sequence | (a.a.) |
| 1 | FAK-Alpha-factor | SEQ ID | MRFPSIFTAVLFAASSALAAPVN | 85 |
| | (S. cerevisiae) | NO: 3 | TTTEDETAQIPAEAVIGYSDLEG | |
| | | | DFDVAVLPFSNSTNNGLLFINTT | |
| | | | IASIAAKEEGVSLEKR | |
| 2 | FAKS-Alpha-factor | SEQ ID | MRFPSIFTAVLFAASSALAAPVN | 89 |
| | full | NO: 4 | TTTEDETAQIPAEAVIGYSDLEG | |
| | (S. cerevisiae) | | DFDVAVLPFSNSTNNGLLFINTT | |
| | | | IASIAAKEEGVSLEKREAEA | |
| 3 | AT-Alpha-factor_T | SEQ ID | MRFPSIFTAVLFAASSALALEKR | 23 |
| | (S. cerevisiae) | NO: 5 | | |
| 4 | AA-Alpha-amylase | SEQ ID | MVAWWSLFLYGLQVAAPALAL | 24 |
| | (Aspergillus niger) | NO: 6 | EKR | |
| 5 | GA-Glucoamylase | SEQ ID | MSFRSLLALSGLVCSGLALEKR | 22 |
| | (Aspergillus awamori) | NO: 7 | | |
| 6 | IN-Inulinase | SEQ ID | MKLAYSLLLPLAGVSALEKR | 20 |
| | (Kluyveromyces | NO: 8 | | |
| | maxianus) | | | |
| 7 | IV-Invertase | SEQ ID | MLLQAFLFLLAGFAAKISALEK | 23 |
| | (S. cerevisiae) | NO: 9 | R | |
| 8 | KP-Killer protein | SEQ ID | MTKPTQVLVRSVSILFFITLLHL | 30 |
| | (S. cerevisiae) | NO: 10 | VVALEKR | |
| 9 | LZ-Lysozyme | SEQ ID | MLGKNDPMCLVLVLLGLTALL | 30 |
| | (Gallus gallus) | NO: 11 | GICQGLEKR | |
| 10 | SA-Serum albumin | SEQ ID | MKWVTFISLLFLFSSAYSLEKR | 22 |
| | (Homo sapiens) | NO: 12 | | |

In all the secretory signal peptide sequences, a stretch of four amino acids (LEKR) was added for the efficient Kex2 processing of pre-protein.

TABLE 2
Modified nucleic acid sequences of β-fructofuranosidase (fopA) gene fused to
signal peptides

| | | | Length |
|---|---|---|---|
| Sr. No. | Description | SEQ ID NO | (b.p.) |
| 1 | FAK—Alpha-factor of S. cerevisiae fused to modified | SEQ ID | 2220 |
| | nucleic acid of β-fructofuranosidase (fopA) gene | NO: 13 | |
| 2 | FAKS—Alpha-factor full of S. cerevisiae fused to modified | SEQ ID | 2232 |
| | nucleic acid of β-fructofuranosidase (fopA) gene | NO: 14 | |
| 3 | AT—Alpha-factor_T of S. cerevisiae fused to modified | SEQ ID | 2034 |
| | nucleic acid of β-fructofuranosidase (fopA) gene | NO: 15 | |
| 4 | AA—Alpha-amylase of Aspergillus niger fused to modified | SEQ ID | 2037 |
| | nucleic acid of β-fructofuranosidase (fopA) gene | NO: 16 | |
| 5 | GA—Glucoamylase of Aspergillus awamori fused to | SEQ ID | 2031 |
| | modified nucleic acid of β-fructofuranosidase (fopA) gene | NO: 17 | |
| 6 | IN—Inulinase of Kluyveromyces maxianus fused to modified | SEQ ID | 2025 |
| | nucleic acid of β-fructofuranosidase (fopA) gene | NO: 18 | |
| 7 | IV—Invertase of S. cerevisiae fused to modified nucleic acid | SEQ ID | 2034 |
| | of β-fructofuranosidase (fopA) gene | NO: 19 | |
| 8 | KP—Killer protein of S. cerevisiae fused to modified nucleic | SEQ ID | 2055 |
| | acid of β-fructofuranosidase (fopA) gene | NO: 20 | |
| 9 | LZ—Lysozyme of Gallus gallus fused to modified nucleic | SEQ ID | 2055 |
| | acid of β-fructofuranosidase (fopA) gene | NO: 21 | |
| 10 | SA—Serum albumin of Homo sapiens fused to modified | SEQ ID | 2031 |
| | nucleic acid of β-fructofuranosidase (fopA) gene | NO: 22 | |

SEQ ID NO: 23—Native nucleic acid sequence of the fopA gene (1965 base pairs) encoding secreted β-fructofuranosidase

TABLE 3
Bioactive fragments of β-fructofuranosidase are
conserved and accounts for the catalytic
activities

| Position | Fragment | SEO ID Number |
|---|---|---|
| 57-62 | QIGDPC | SEQ ID NO: 24 |
| 119-132 | DGAVIPVGVNNTPT | SEQ ID NO: 25 |
| 320-330 | SGLPIVPQVS | SEQ ID NO: 26 |
| 401-416 | GDQYEQADGFPTAQQG | SEQ ID NO: 27 |

### Definitions

Unless defined otherwise, all technical and scientific terms used herein have the same meaning as commonly understood by one of ordinary skill in the art to which the methods belong. Although any vectors, host cells, methods and compositions similar or equivalent to those described herein can also be used in the practice or testing of the vectors, host cells, methods and compositions, representative illustrations are now described.

Where a range of values are provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods and compositions.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

The term “host cell(s)” includes an individual cell or cell culture which can be, or has been, a recipient for the subject of expression constructs. Host cells include progeny of a single host cell. Host cells for the purposes of this invention refers to any strain of Pichia pastoris which can be suitably used for the purposes of the invention. Examples of strains that can be used for the purposes of this invention include wild type, mut+, mut S, mut− strains of Pichia such as KM71H, KM71, SMD1168H, SMD 1168, GS115, X33.

The term “recombinant strain” or “recombinant host cell(s)” refers to a host cell(s) which has been transfected or transformed with the expression constructs or vectors of this invention.

The term “expression vector” refers to any vector, plasmid or vehicle designed to enable the expression of an inserted nucleic acid sequence following transformation into the host.

The term “promoter” refers to DNA sequences that define where transcription of a gene begins. Promoter sequences are typically located directly upstream or at the 5′ end of the transcription initiation site. RNA polymerase and the necessary transcription factors bind to the promoter sequence and initiate transcription. Promoters can either be constitutive or inducible promoters. Constitutive promoters are the promoter which allows continual transcription of its associated genes as their expression is normally not conditioned by environmental and developmental factors. Constitutive promoters are very useful tools in genetic engineering because constitutive promoters drive gene expression under inducer-free conditions and often show better characteristics than commonly used inducible promoters. Inducible promoters are the promoters that are induced by the presence or absence of biotic or abiotic and chemical or physical factors. Inducible promoters are a very powerful tool in genetic engineering because the expression of genes operably linked to them can be turned on or off at certain stages of development or growth of an organism or in a particular tissue or cell type.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter).

The term “transcription” refers to the process of making an RNA copy of a gene sequence. This copy, called a messenger RNA (mRNA) molecule, leaves the cell nucleus and enters the cytoplasm, where it directs the synthesis of the protein, which it encodes.

The term “translation” refers to the process of translating the sequence of a messenger RNA (mRNA) molecule to a sequence of amino acids during protein synthesis. The genetic code describes the relationship between the sequence of base pairs in a gene and the corresponding amino acid sequence that it encodes. In the cell cytoplasm, the ribosome reads the sequence of the mRNA in groups of three bases to assemble the protein.

The term “expression” refers to the biological production of a product encoded by a coding sequence. In most cases, a DNA sequence, including the coding sequence, is transcribed to form a messenger-RNA (mRNA). The messenger-RNA is then translated to form a polypeptide product that has a relevant biological activity. Also, the process of expression may involve further processing steps to the RNA product of transcription, such as splicing to remove introns, and/or post-translational processing of a polypeptide product.

The term “modified nucleic acid” as used herein is used to refer to a nucleic acid encoding β-fructofuranosidase fused to a signal peptide. In embodiments, the modified nucleic acid is represented by SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO: 21, SEQ ID NO: 22 or a functionally equivalent variant thereof. The functional variant includes any nucleic acid having substantial or significant sequence identity or similarity to SEQ ID NO:13-22, and which retains the biological activities of the same.

The terms “polypeptide”, “peptide” and “protein” are used interchangeably herein to refer to two or more amino acid residues joined to each other by peptide bonds or modified peptide bonds. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. “Polypeptide” refers to both short chains, commonly referred to as peptides, oligopeptides or oligomers, and to longer chains, generally referred to as proteins. Polypeptides may contain amino acids other than the 20 gene-encoded amino acids. Likewise, “protein” refers to at least two covalently attached amino acids, which includes proteins, polypeptides, oligopeptides, and peptides. A protein may be made up of naturally occurring amino acids and peptide bonds, or synthetic peptidomimetic structures. Thus “amino acid”, or “peptide residue”, as used herein means both naturally occurring and synthetic amino acids. “Amino acid” includes imino acid residues such as proline and hydroxyproline. The side chains may be in either the (R) or the (S) configuration.

The term “signal peptide” or “signal peptide sequence” is defined herein as a peptide sequence usually present at the N-terminal end of newly synthesized secretory or membrane polypeptides which directs the polypeptide across or into a cell membrane of the cell (the plasma membrane in prokaryotes and the endoplasmic reticulum membrane in eukaryotes). It is usually subsequently removed. In particular said signal peptide may be capable of directing the polypeptide into a cell's secretory pathway.

The term “precursor peptide” as used herein refers to a peptide comprising a signal peptide (also known as leader sequences) operably linked to the β-fructofuranosidase of Aspergillus niger. The signal peptides are cleaved off during post-translational modifications inside the Pichia host cells and the mature β-fructofuranosidase (SEQ ID NO: 1) is released into the medium.

The term “variant” as used herein in reference to pre-cursor peptides/proteins refers to peptides with amino acid substitutions, additions, deletions or alterations that do not substantially decrease the activity of the signal peptide or the enzyme. Variants include a structural as well as functional variants. The term variant also includes the use of a substituted amino acid in place of an unsubstituted parent amino acid.

Amino acid substitution tables providing functionally similar amino acids are well known to one of ordinary skill in the art. The following six groups are examples of amino acids that are considered to be variants for one another:

TABLE 4
Amino acid substitution table

| | Amino acids |
|---|---|
| Group 1 | Alanine (A), Serine (S), Threonine (T), Glycine (G), |
| | Proline (P) |
| Group 2 | Aspartic acid (D), Glutamic acid (E), Asparagine (N), |
| | Glutamine (Q) |
| Group 3 | Arginine (R), Lysine (K), Histidine (H) |
| Group 4 | Isoleucine (I), Leucine (L), Methionine (M), Valine (V) |
| Group 5 | Phenylalanine (F), Tyrosine (Y), Tryptophan (W) |
| Group 6 | Cysteine (C) |

### DETAILED DESCRIPTION OF THE INVENTION

The present invention discloses nucleic acids, vectors and recombinant host cells for efficient production of biologically active and soluble recombinant β-fructofuranosidase of Aspergillus niger as a secreted protein. Further, the invention provides a process for commercial-scale production of recombinant β-fructofuranosidase.

The invention contemplates a multidimensional approach for achieving a high yield of novel recombinant β-fructofuranosidase in a heterologous host. The native gene for β-fructofuranosidase has been modified for expression in Pichia pastoris. Further, the modified gene has been fused to one or more signal peptides.

In one embodiment, the modified nucleic acid encoding novel 1-fructofuranosidase of Aspergillus niger is represented by SEQ ID NO: 2.

In another embodiment, the modified nucleic acid is fused to one or more signal peptide.

In another embodiment, the signal peptide is selected from Alpha-factor of S. cerevisiae (FAK), Alpha-factor full of S. cerevisiae (FAKS) of S. cerevisiae, Alpha factor_T of S. cerevisiae (AT), Alpha-amylase of Aspergillus niger (AA), Glucoamylase of Aspergillus awamori (GA), Inulinase of Kluyveromyces maxianus (IN), Invertase of S. cerevisiae (IV), Killer protein of S. cerevisiae (KP), Lysozyme of Gallus gallus (LZ), Serum albumin ofHomo sapiens (SA)

In another embodiment, the signal peptide are provided in the below Table 5.

TABLE 5
Signal peptides

| Sr. | Signal Peptides | | Length |
|---|---|---|---|
| No. | (Source) | Amino Acid Sequence | (a.a.) |
| 1 | FAK-Alpha-factor | MRFPSIFTAVLFAASSALAAPVNTTTEDE | 81 |
| | (S. cerevisiae) | TAQIPAEAVIGYSDLEGDFDVAVLPFSNS | |
| | | TNNGLLFINTTIASIAAKEEGVS | |
| 2 | A-Alpha-factor_T | MRFPSIFTAVLFAASSALA | 19 |
| | (S. cerevisiae) | | |
| 3 | AA-Alpha-amylase | MVAWWSLFLYGLQVAAPALA | 20 |
| | (Aspergillus niger) | | |
| 4 | GA-Glucoamylase | MSFRSLLALSGLVCSGLA | 18 |
| | (Aspergillus awamori) | | |
| 5 | IN-Inulinase | MKLAYSLLLPLAGVSA | 16 |
| | (Kluyveromyces | | |
| | maxianus) | | |
| 6 | IV-Invertase | MLLQAFLFLLAGFAAKISA | 19 |
| | (S. cerevisiae) | | |
| 7 | KP-Killer protein | MTKPTQVLVRSVSILFFITLLHLVVA | 26 |
| | (S. cerevisiae) | | |
| 8 | LZ-Lysozyme | MLGKNDPMCLVLVLLGLTALLGICQG | 26 |
| | (Gallus gallus) | | |
| 9 | SA-Serum albumin | MKWVTFISLLFLFSSAYS | 18 |
| | (Homo sapiens) | | |

In another embodiment, the signal peptide is selected from a list of modified signal peptides as described in Table 1.

In another embodiment, the nucleic acid fused to one or more modified signal peptide selected from a group comprising SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and variants thereof.

In another embodiment, the modified nucleic acid is cloned in an expression vector.

In another embodiment, the expression vector is configured for secretory or intracellular expression of recombinant β-fructofuranosidase from Aspergillus niger.

In yet another embodiment, the expression vector is selected from a group comprising pPICZαA, pPICZαB, pPICZαC, pGAPZαA, pGAPZαB, pGAPZαC, pPIC3, pPIC3.5, pPIC3.5K, PAO815, pPIC9, pPIC9K, IL-D2 and pHIL-S1.

The expression of the modified β-fructofuranosidase (fopA) gene fused to a signal peptide is preferably driven by a constitutive or inducible promoter.

In another embodiment, the nucleic acid to be expressed in operably linked to the promoter.

In another embodiment, the constitutive or inducible promoter is selected from a group listed in Table 6.

TABLE 6
List of promoters used

| | Promoter | Gene | | | Expression |
|---|---|---|---|---|---|
| Sr. No. | Type | Name | Gene Product | Inducer | Level |
| 1 | Inducible | AOX1 | Alcohol oxidase 1 | Methanol | Strong |
| 2 | Inducible | ADH3 | Alcohol dehydrogenase | Ethanol | Strong |
| 3 | Inducible | DAS | Dihyroxyacetone phosphate | Methanol | Strong |
| 4 | Inducible | FLD1 | Formaldehyde dehydrogenase | Methanol/ | Strong |
| | | | | Methylamine | |
| 5 | Inducible | LRA3 | L-rhamnonate dehydratase | Rhamnose | 75% of |
| | | | | | pGAP |
| 6 | Inducible | THI11 | Thiamine Biosynthesis | Repressed by | 70% of |
| | | | Protein | Thiamine | pGAP |
| 7 | Constitutive | GAP | Glyceraldehyde 3- | — | strong |
| | | | phosphatedehydrogenate | | |
| 8 | Constitutive | YPT1 | GPTase involved in sectetion | — | weak |
| 9 | Constitutive | TEF1 | Translation elongation factor 1 alpha | — | strong |
| 10 | Constitutive | GCW14 | Glycosylphosphatidylinositol | — | strong |
| 11 | Constitutive | PGK1 | Phosphoglycerate kinase | — | 10% of |
| | | | | | pGAP |

In another embodiment, the promoter is an AOX1 promoter, which is induced by methanol and repressed by glucose.

In an embodiment, the expression vector containing the modified gene of interest (β-fructofuranosidase gene fused to a nucleic acid encoding signal peptide) is transformed in an appropriate host.

In another embodiment, the expression vector containing the gene of interest is transformed in yeast cells.

In another embodiment, the yeast cell is a Pichia pastoris.

In yet another embodiment, the Pichia Pastoris host cell is a mut+, mut S or mut-strains. Mut+ represents methanol utilization plus phenotype.

In yet another embodiment, the Pichia Pastoris host cell strain is selected from a group comprising KM71H, KM71, SMD 1168H, SMD 1168, GS 115, X33.

In another embodiment, the invention provides β-fructofuranosidase pre-cursor peptides, wherein β-fructofuranosidase of Aspergillus niger is fused to one or more signal peptide.

In another embodiment, β-fructofuranosidase of Aspergillus niger has the amino acid sequence set forth in SEQ ID NO:1 and functional variants thereof. Functional variant includes any protein sequence having substantial or significant sequence identity or similarity to SEQ ID NO:1 and or having a substantial or significant structural identity or similarity to SEQ ID NO:1, and which retains the biological activities of the same.

In another embodiment, the signal peptide is selected from a group comprising Alpha-factor full of S. cerevisiae (FAK) set forth in SEQ ID NO: 3, Alpha-factor full of S. cerevisiae (FAKS) set forth in SEQ ID NO: 4, Alpha factor_T of S. cerevisiae (AT) set forth in SEQ ID NO: 5, Alpha-amylase of Aspergillus niger (AA) set forth in SEQ ID NO: 6, Glucoamylase of Aspergillus awamori (GA) set forth in SEQ ID NO: 7, Inulinase of Kluyveromyces maxianus (IN) set forth in SEQ ID NO: 8, Invertase of S. cerevisiae (IV) set forth in SEQ ID NO: 9, Killer protein of S. cerevisiae (KP) set forth in SEQ ID NO: 10, Lysozyme of Gallus gallus (LZ) set forth in SEQ ID NO: 11, Serum albumin of Homo sapiens (SA) set forth in SEQ ID NO: 12, and variants thereof.

In an embodiment, the process for the production of recombinant β-fructofuranosidase of Aspergillus niger is provided.

Aspects of the present invention relate to fermentation of recombinant Pichia pastoris cells containing modified recombinant β-fructofuranosidase (fopA) gene. After completion of the fermentation, the fermentation broth is subjected to centrifugation and filtered using microfiltration and the recombinant enzyme is separated. The recovered recombinant enzyme is concentrated using Tangential Flow Ultra-filtration or evaporation and finally the concentrated enzyme is formulated.

In one embodiment, the process for expressing β-fructofuranosidase of Aspergillus niger at high levels comprises the steps of:
a. culturing recombinant host cells in a suitable fermentation medium to obtain recombinant β-fructofuranosidase enzyme secreted into fermentation broth;
b. harvesting supernatant from the fermentation broth, wherein the supernatant contains recombinant β-fructofuranosidase; and
c. purifying recombinant β-fructofuranosidase.

In another embodiment, the fermentation medium is basal salt medium as described in Table 7.

In yet another embodiment, the supernatant from the fermentation broth is harvested using centrifugation.

In one embodiment, the percentage of inoculum or starter culture to initiate the fermenter culture is in the range of 2.0% to 15.0% (v/v).

In another embodiment, the pH of the fermentation medium is maintained in the range of 4.0 to 7.5 as the secreted enzyme undergoes proper folding and is biologically active at this pH range.

In yet another embodiment, the temperature of the fermentation process is in the range of 15° C. to 40° C.

In another embodiment, the time for fermentation process is in the range of 50-150 hrs.

In a further, embodiment, the fermentation broth is centrifuged at a speed in the range from 2000×g to 15000×g using continuous online centrifugation.

The supernatant obtained after centrifugation is subjected to microfiltration and purified to recover biologically active recombinant β-fructofuranosidase.

In one embodiment, the supernatant obtained after centrifugation is concentrated using a Tangential Flow Filtration based Ultra filtration System.

The cut-off size of the membranes used in Tangential Flow Filtration (TFF) systems that may be used to remove impurities and to concentrate the collected culture supernatant may range between 5 to 100 kDa.

In another embodiment, no centrifugation is required for the process due to the high yield and purity of the secreted enzyme.

The β-fructofuranosidase concentration obtained in this invention is found to be in the range of 2-5 gm/L and the purity is about 85%.

### EXAMPLES

The following examples particularly describe the manner in which the invention is to be performed. But the embodiments disclosed herein do not limit the scope of the invention in any manner.

### Example 1: Modified Nucleic Acids for Expression of Recombinant β-Fructofuranosidase of Aspergillus niger in Pichia pastoris

The cDNA of the native β-fructofuranosidase (fopA) of Aspergillus niger is represented by SEQ ID NO: 23 and the amino acid sequence of novel β-fructofuranosidase is represented by SEQ ID NO: 1.

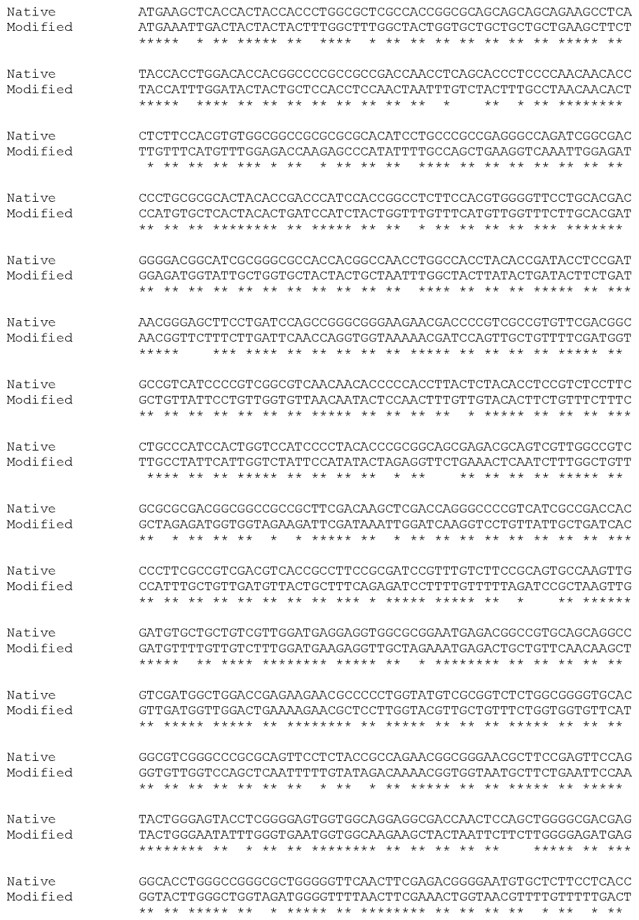

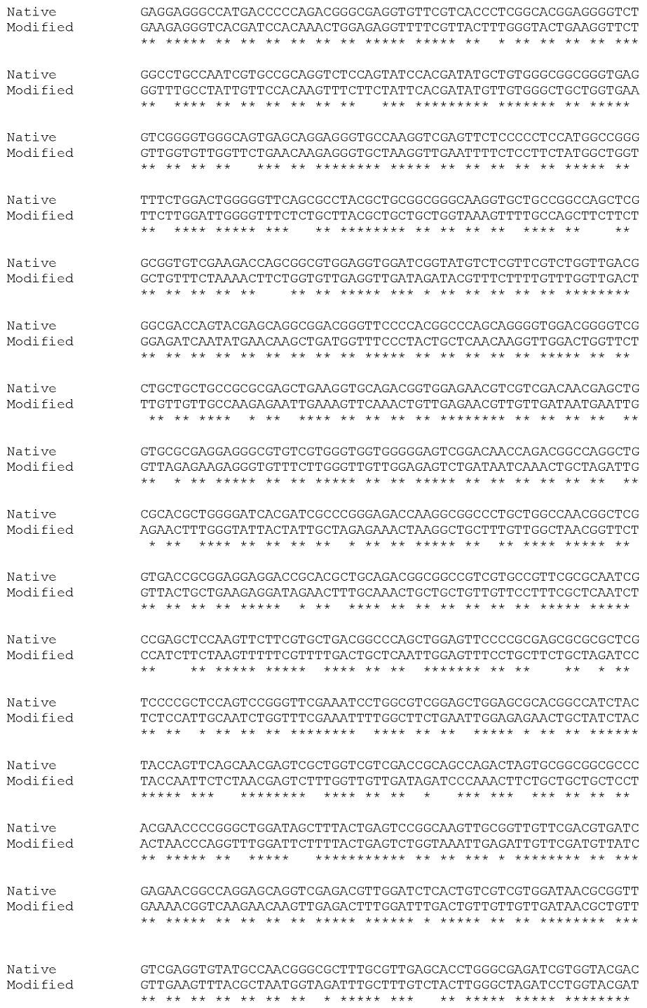

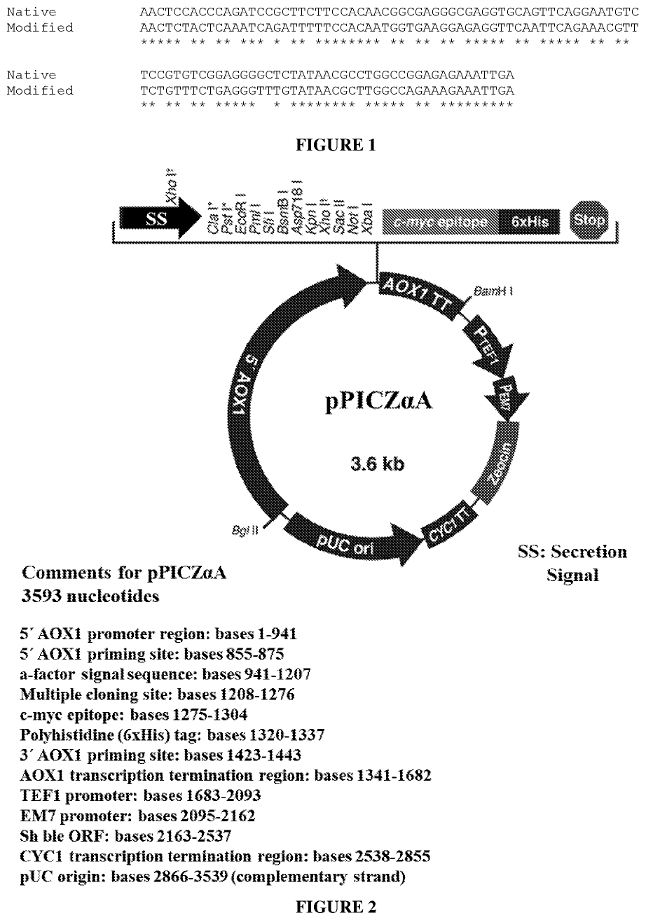

The native cDNA was modified for maximizing expression in Pichia pastoris. The modified nucleic acid is represented by SEQ ID NO: 2. The differences between the native and the modified sequence is depicted in FIG. 1.

An expression cassette encoding the β-fructofuranosidase was modified for maximizing expression in Pichia pastoris. The modified open reading frame contains the modified nucleotide sequence (SEQ ID NO: 2) encoding β-fructofuranosidase fused to a signal peptide. The nucleic acids have been designed such that the encoded signal peptides contain an additional stretch of four amino acids (LEKR) for the efficient Kex2 processing of precursor peptide.

The preferred codons for expression in Pichia pastoris have been used in place of rare codons.

The nucleotide sequence of the modified open reading frames encoding for β-fructofuranosidase fused with modified signal peptides are given below:
Alpha-factor of S. cerevisiae (FAK) is represented by SEQ ID NO: 13
Alpha-factor full of S. cerevisiae (FAKS) is represented by SEQ ID NO: 14
Alphafactor_T of S. cerevisiae (AT) represented by SEQ ID NO: 15
Alpha-amylase of Aspergillus niger (AA) represented by SEQ ID NO: 16
Glucoamylase of Aspergillus awamori (GA) represented by SEQ ID NO: 17
Inulinase of Kluyveromyces maxianus (IN) represented by SEQ ID NO: 18
Invertase of S. cerevisiae (IV) represented by SEQ ID NO: 19
Killer protein of S. cerevisiae (KP) represented by SEQ ID NO: 20
Lysozyme of Gallus gallus (LZ) represented by SEQ ID NO: 21
Serum albumin of Homo sapiens (SA) represented by SEQ ID NO: 22.

The SEQ ID NO: 13 nucleic acid sequence was chemically synthesized cloned into pPICZαA vector and remaining modified nucleic acid sequences have been generated by overlap extension PCR using SEQ ID NO: 13 expression cassette as a template.

### Example 2: Polypeptide Sequences of β-Fructofuranosidase Fused to Signal Peptides

Recombinant pre-cursor proteins were obtained by translating the gene encoding for β-fructofuranosidase of Aspergillus niger fused with signal peptides.

The signal peptides used in the modified precursor peptides were Alpha-factor of S. cerevisiae (FAK) represented by SEQ ID NO: 3, Alpha-factor full of S. cerevisiae (FAKS) represented by SEQ ID NO: 4, Alpha-factor_T of S. cerevisiae (AT) represented by SEQ ID NO: 5, Alpha-amylase of Aspergillus niger (AA) represented by SEQ ID NO: 6, Glucoamylase of Aspergillus awamori (GA) represented by SEQ ID NO: 7, Inulinase of Kluyveromyces maxianus (IN) represented by SEQ ID NO: 8, Invertase of S. cerevisiae (IV) represented by SEQ ID NO: 9, Killer protein of S. cerevisiae (KP) represented by SEQ ID NO: 10, Lysozyme of Gallus gallus (LZ) represented by SEQ ID NO: 11 and Serum albumin of Homo sapiens (SA) represented by SEQ ID NO: 12. The modified signal peptides contain an additional stretch of four amino acids (LEKR) for the efficient Kex2 processing of precursor peptide.

The signal peptides are cleaved off during post-translational modifications inside the Pichia host cells and the mature recombinant β-fructofuranosidase comprising the amino acid sequence of SEQ ID NO: 1 is released into the medium.

### Example 3: Development of Recombinant Host Cells by Transformation with Recombinant Plasmids

The vector used in the process was pPICZαA. The vectors contained the modified open reading frames as described in Example 1 and an inducible promoter, AOX). The modified sequence encoding for the recombinant protein was cloned into the pPICZαA vector.

The modified nucleic acid SEQ ID NO: 2 encoding f-fructofuranosidase (fopA) gene was cloned between XhoI/SacII restriction sites present in the MCS of pPICZαA vector to bring signal sequence Alpha-factor of S. cerevisiae (FAK) in frame to create SEQ ID NO: 13 expression cassette using regular molecular biology procedures. The vector map for pPICZαA is represented in FIG. 2.

The putative recombinant plasmids were selected on low salt-LB media containing 25 μg/ml Zeocin and screened by XhoI/SacII restriction digestion analysis.

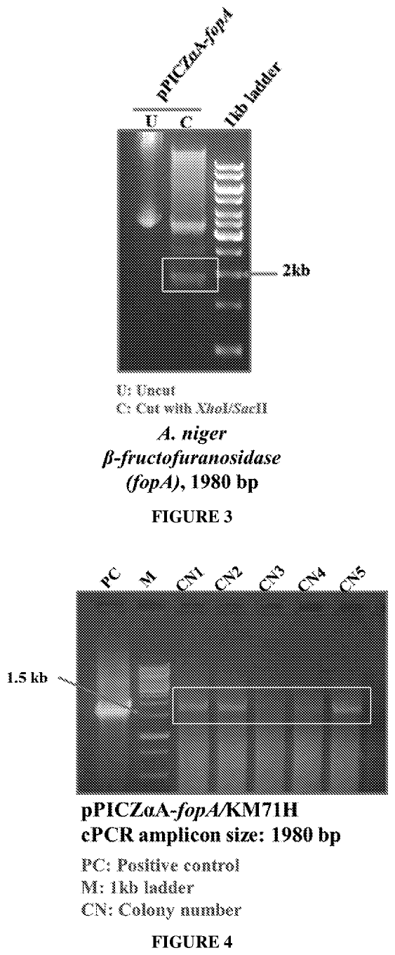

The recombinant plasmid pPICZαA-fopA was confirmed by XhoI/SacII restriction digestion analysis which resulted in release of 1980 bp fragment. The results of the restriction digestion analysis are depicted in FIG. 3.

Thereafter, Pichia pastoris KM71H cells were electroporated with linearized recombinant pPICZαA-fopA DNA. The Pichia integrants were selected on yeast extract peptone dextrose sorbitol agar (YPDSA) containing 100 μg/ml Zeocin.

The integration was screened with colony PCR (cPCR). For cPCR, a template from each of the Pichia integrants was generated by the alkali lysis method. The results of the colony PCR screening are depicted in FIG. 4.

The Pichia integrants were grown for 48 h in BMD1 media and further induced first with BMM2 and then successively with BMM10 media which provided final concentration of 0.5% methanol in the culture medium. At the end of 96 hrs induction period, culture supernatants from different clones were harvested. Total protein from each of the harvested supernatants was precipitated with 20% TCA and analyzed on SDS-PAGE.

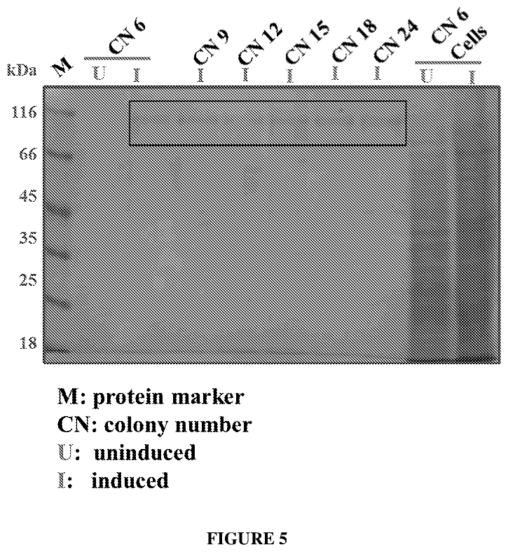

Upon induction β-fructofuranosidase protein bands were seen at the size of approximately 110 kDa as depicted in FIG. 5.

The calculated molecular weight was about 70.85 kDa. The increase in molecular weight may have been contributed by glycosylation.

### Example 4: Fermentation of Recombinant Pichia pastoris Expressing β-Fructofuranosidase of Aspergillus niger

Fermentation of recombinant Pichia pastoris cells containing the modified β-fructofuranosidase (fopA) gene as described in Example 1 was carried out in a 50 L fermenter. Fermentation was carried out in basal salt medium as described herein. The recombinant host selected was KM71H, which is a mut S strain that metabolizes methanol in a slow manner.

### Preparation of Pre-Seed and Seed Inoculum:

The pre-seed was generated by inoculating from the glycerol stock in 25 mL of sterile YEPG medium and growing at 30° C. in a temperature-controlled orbital shaker overnight. For generating seed, the inoculum was grown in Basal salt medium in baffled shake flasks at 30° C. in a temperature-controlled orbital shaker till OD₆₀₀ of 15-25 was reached.

### Fermentation Process

The entire process of fermentation from the inoculation of fermenter with seed culture to final harvesting took about 130 hrs. Basal salt medium was prepared and sterilized in situ in the fermenter.

The composition of basal salt medium optimized for the fermentation process is provided in Table 7.

TABLE 7
Composition of basal salt medium

| Component | Concentration |
|---|---|
| Calcium Sulphate | 1.4 gm/L |
| Potassium Sulphate | 18.6 gm/L |
| Magnesium Sulphate•7H₂O | 16.4 gm/L |
| Glycerol | 25 gm/L |
| Potassium Di hydrogen Phosphate | 5 gm/L |
| Ammonium Sulphate | 5 mL |
| Sodium Citrate Di Hydrate | 5 gm/L |
| PTM2 | 4 mL |
| Biotin (20 mg/100 ml) | 4 mL |

Pichia Trace Minerals (PTM) salt solution was prepared as described in Table 8. PTM salts were dissolved and made up to 1 L volume and filter sterilized. PTM salt solution was included at the rate of 4 ml per liter of initial media volume after sterilization of the basal salt media

TABLE 8
PTM trace salts

| | |
|---|---|
| Cupric sulfate•5H₂O | 2.0 gm/L |
| Sodium iodide | 0.08 gm/L |
| Manganese sulfate•H₂O | 3.0 gm/L |
| Sodium molybdate•2H₂O | 0.2 gm/L |
| Boric Acid | 0.02 gm/L |
| Cobalt chloride | 0.5 gm/L |
| Zinc Sulphate | 7.0 gm/L |
| Ferrous sulfate•7H₂O | 22.0 gm/L |
| Potassium chloride | 0.37 gm/L |
| Sulfuric Acid | 1 mL |
| Ferric chloride | 0.811 gm/L |
| Nickel chloride | 1.18 gm/L |
| Magnesium sulfate | 1.23 gm/L |

### Growth Phase:

The growth phase starts by inoculating basal salt medium in 50 L fermenter with 5% seed culture and continues for about 24 hours. The dissolved oxygen (DO) levels were continuously monitored and never allowed to drop below 40%.

After 18 h, a DO spike was observed indicating the depletion of carbon source (Glycerol). A glycerol fed-batch was initiated by feeding 50% Glycerol (with 12 ml of PTM salts per liter of feed) for about six hours till the OD₆₀₀ reached 200.

### Induction Phase:

Once sufficient biomass was generated, the induction phase was initiated by discontinuing glycerol feed and starting methanol feed. Methanol (supplemented with 12 ml of PTM salts per liter of feed) was fed at the rate of 0.5 g to 3 g per liter of initial fermentation volume. The DO was maintained at 40% and methanol feed was accordingly adjusted.

The induction of β-fructofuranosidase (fopA) gene was monitored periodically by analyzing culture supernatant by enzyme activity assay. The induction phase was continued for about 100 hours till the OD₆₀₀ reached 600 and wet biomass reached ˜560 grams per liter of culture broth.

The fermentation was stopped after 130 hours and enzyme activity in the fermenter broth at the end of fermentation was determined to be 10573 units by DNS method (Miller, 1959). One unit is defined as the amount of enzyme required to release one micromole of reducing sugars (glucose equivalents) from 10% sucrose solution in 100 mM citrate buffer pH 5.5 at 55° C. The total amount of recombinant β-fructofuranosidase in the culture broth was estimated by Bradford assay.

### Fermentation Conditions:

The fermentation parameters considered were as given in Table 9. These essential parameters were monitored during the fermentation process.

TABLE 9
Fermentation Parameters

| Fermentation parameters | Growth phase | Induction phase |
|---|---|---|
| Media | Basal Salt Media | Basal Salt Media |
| pH | 5 | 5 |
| Temperature | 30 | 25 |
| Agitation (tip speed) | 1.2-2.5 m/Sec | 2.5 m/Sec |
| Aeration | 0.5-1.5 vvm | 1.5 vvm |
| Dissolved oxygen | Minimum 40% | Minimum 40% |
| Back pressure | 0.5 kg/cm² | 0.5 kg/cm² |

### Example 5: Cell Harvesting and Purification

Harvesting of the enzyme is performed by continuous centrifugation at 8000 RPM. Clear supernatant obtained after centrifugation was subjected to microfiltration using 0.1 microns cut off spiral wound TFF membrane. The filtrate is further subjected to ultrafiltration and diafiltration using 10 kDa cutoff spiral wound TFF membrane and sufficiently concentrated and to reach the desired activity. The enzyme was formulated by including 35-50% of glycerol and food-grade preservatives in the final preparation. The final purity of the enzyme was observed to be 85% as determined by SDS-PAGE analysis.

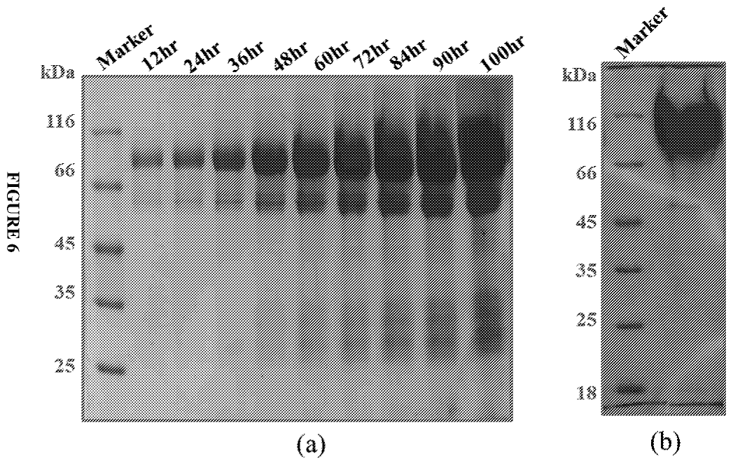

FIG. 6 (a) depicts the SDS-PAGE analysis of samples collected at different time intervals during fermentation of Pichia pastoris KM71H strain expressing recombinant β-fructofuranosidase enzyme. FIG. 6 (b) depicts the SDS-PAGE analysis of recombinant β-fructofuranosidase enzyme after purification.

The β-fructofuranosidase concentration was found to be about 2.4 gm/L. In most of the batches, the concentration was 2-5 gm/L. The purity of the recombinant β-fructofuranosidase was observed to be about 85%.

### Example 6: Estimation of β-Fructofuranosidase Activity

Studies were conducted to estimate the activity of β-fructofuranosidase. For the estimation studies, the amount of reducing sugar generated due to the action of β-fructofuranosidase enzyme was calculated using DNS (3,5 Dinitrosalicylic acid) method (G. L. Miller, “Use of dinitrosalicylic acid reagent for determination of reducing sugar”, Anal. Chem., 1959, 31, 426-428).

For conducting the enzyme activity assay, 10% Sucrose (dissolved in 100 mM Citrate buffer) was used as the substrate. β-fructofuranosidase was recovered from the fermentation broth and processed through ultra-filtration. The ultra-filtered sample then diluted 25,000× by serial dilution in 100 mM Citrate buffer and was used. The reaction volume was 2.5 mL. The pH was maintained at 5.5 and the reaction was continued for 15 minutes.

After incubation 3 mL of DNS (3,5 Dinitrosalicylic acid) was added to each reaction mixture and boiled for 10 min, cooled and read absorbance at 540 nm, spectrophotometrically.

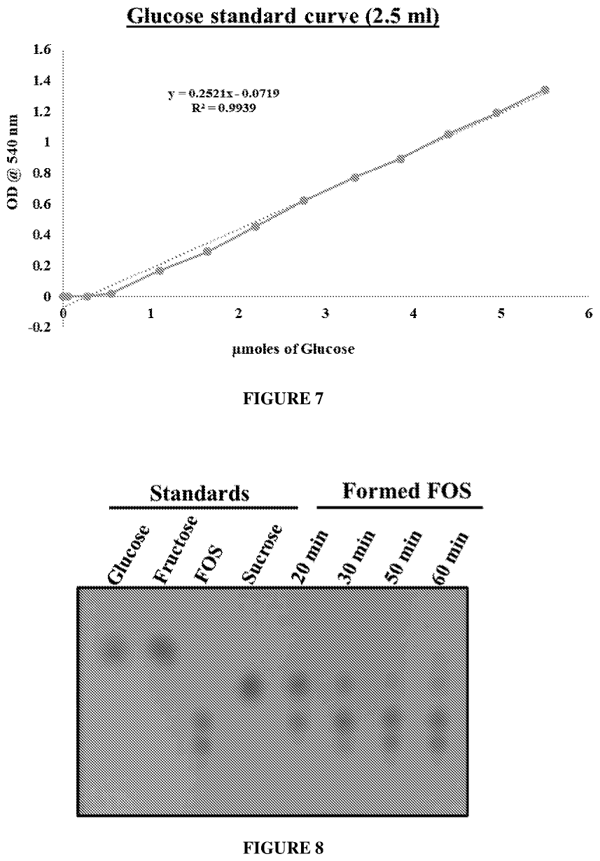

The OD of glucose at different concentration was measured as shown in Table 10 and depicted in FIG. 7. Thereafter, based on the absorbance measurement after the reaction, the enzyme activity was calculated as shown in Table 11. FIG. 7 depicts the Glucose standard curve used for the estimation of the activity of β-fructofuranosidase enzyme.

TABLE 10
OD measurement of glucose at different concentration

| Glucose(μmol) | OD at 540 nm | Glucose(μmol) | OD at 540 nm |
|---|---|---|---|
| 0 | 0 | 2.75 | 0.619 |
| 0.055 | 0 | 3.33 | 0.77 |
| 0.55 | 0.018 | 3.85 | 0.891 |
| 1.1 | 0.165 | 4.44 | 1.052 |
| 1.65 | 0.289 | 4.95 | 1.198 |
| 2.2 | 0.452 | 5.5 | 1.338 |

TABLE 11
Estimation of activity of β-fructofuranosidase

| Reaction | Buffer | Substrate | Enzyme | OD @ | Effective | Unit/ |
|---|---|---|---|---|---|---|
| test tubes | (mL) | (mL) | (mL) | 540 nm | OD | mL |
| Reagent | 2.5 | — | — | 0.000 | — | — |
| blank | | | | | | |
| Substrate | 0.1 | 2.4 | — | 0.230 | — | — |
| blank | | | | | | |
| Enzyme | 2.4 | — | 0.1 (25,000X | 0.000 | — | — |
| blank | | | diluted) | | | |
| Enzyme | — | 2.4 | 0.1 (25,000X | 0.940 | 0.71 | 51692 |
| Reaction | | | diluted) | | | |

### Example 7: Generation of Fructooligosaccharides (FOS) from Sucrose and Recombinant β-Fructofuranosidase Enzyme

Studies were conducted to understand the ability of the enzyme in the formation of fructooligosaccharides. A 100 ml, solution of 90% (w/v) Sucrose was prepared in 150 mM sodium citrate buffer pH 5.5. To this, 96.7 μL of β-fructofuranosidase enzyme having 51692 Unit/ml of activity (equivalent to total of 5000 Units of enzyme), was added.

The reaction was set up in a 250 ml, conical flask and incubated at 65° C. and 220 rpm. At regular time intervals, samples were taken and analyzed on Thin Layer Chromatographic (TLC) plates.

Glucose, sucrose, fructose and FOS (containing kestose, nystose and fructofuranosylnystose) were used as standards for the thin layer chromatographic analysis. The mobile phase used was n-Butanol:Glacial acetic acid:Water (4:2:2 v/v) and the developing/staining solution used was urea phosphoric acid.

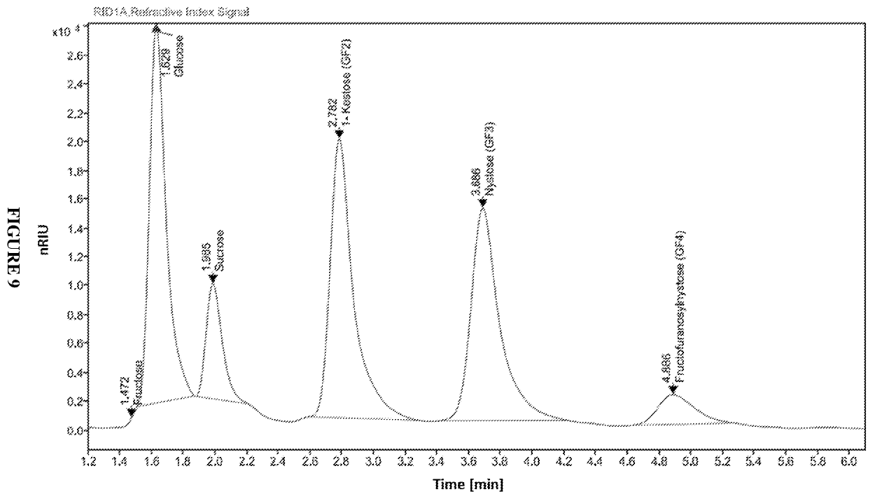

FIG. 8 depicts the TLC analysis done for the generation of fructooligosaccharides (FOS) from sucrose and recombinant β-fructofuranosidase enzyme.

The sample was further subjected to High Performance Liquid Chromatography (HPLC) for quantitative estimation of the production of fructooligosaccharides. The HPLC analysis was done using an amine column (Zorbax NH₂ column, Agilent Technologies) having 4.6 (ID)×150 mm (length) and 5 μm (particle size). The standard solutions of glucose, fructose, kestose, nystose, fructofuranosylnystose and sucrose of different concentrations were run for generating standard curves.

FIG. 9 depicts the HPLC analysis chromatogram of FOS samples. Table 12 depicts the percentage of formation of fructooligosaccharides (FOS) and the recovered glucose, fructose and sucrose at the end of 60 min reaction time.

TABLE 12
The percentage of formation of fructooligosaccharides
(FOS) and the recovered sucrose, glucose
and fructose at the end of 60 min reaction time

| | 90% | On 100% |
|---|---|---|
| | Sucrose substrate | Sucrose substrate basis |
| FOS (%) | 60.5566 | 67.3689 |
| Sucrose (%) | 4.88637 | 5.4360 |
| Glucose (%) | 24.4437 | 27.1934 |
| Fructose (%) | 0.00141 | 0.0015 |

100 ml of 90% (w/v) sucrose solution was reacted with β-fructofuranosidase enzyme for the conversion of sucrose into FOS. The quantities of recovered FOS, sucrose, glucose, and fructose from the reaction after terminating the reaction by heat at the end of 60 min were measured and presented as 90% and 100% sucrose basis.

The studies demonstrated that the purified enzymes are able to effectively convert a very high amount of sugars into fructooligosaccharides.

### Example 8: Characterization of Recombinant β-Fructofuranosidase of Aspergillus niger

The harvested β-fructofuranosidase of Aspergillus niger was characterized to identify bioactive fragments. It was found that following bioactive fragments of β-fructofuranosidase are conserved and accounts for the catalytic activities:

TABLE 13
Bioactive fragments of β-fructofuranosidase are
conserved and accounts for the catalytic
activities

| Position | Fragment | SEq ID Number |
|---|---|---|
| 57-62 | QIGDPC | SEQ ID NO: 24 |
| 119-132 | DGAVIPVGVNNTPT | SEQ ID NO: 25 |
| 320-330 | SGLPIVPQVS | SEQ ID NO: 26 |
| 401-416 | GDQYEQADGFPTAQQG | SEQ ID NO: 27 |

It was further found that the following amino acids residues in β-fructofuranosidase of Aspergillus niger were involved in forming a hydrogen bond network around the catalytic triad. The hydrogen bond network is important for the stable stereochemistry around the catalytic triad:
Arg-190
Tyr-369
Glu-318
His-332
Asp-191
Thr-293
Asp-119
His-144
   It was also found that the following hydrophobic residues in β-fructofuranosidase of Aspergillus niger take part in forming a negatively charged pocket around the active site:
Leu-78
Phe-118
Ala-370
Trp-398
Ile-143
   Further, the following important residues of β-fructofuranosidase of Aspergillus niger that take part in interactions at the entrance of active pocket were identified:
Glu-405
His-332
Tyr-404

1. A modified polypeptide, wherein the polypeptide is β-fructofuranosidase of Aspergillus niger comprising the amino acid sequence of SEQ ID NO: 1 fused to a signal peptide selected from a group comprising FAK, FAKS, AT, AA, GA, IN, IV, KP, LZ and SA or variants thereof.

2. The modified polypeptide as claimed in claim 1, wherein:
a. FAK comprising the amino acid sequence of SEQ ID NO: 3 or variants thereof;
b. FAKS comprising the amino acid sequence of SEQ ID NO: 4 or variants thereof;
c. AT comprising the amino acid sequence of SEQ ID NO: 5 or variants thereof;
d. AA comprising the amino acid sequence of SEQ ID NO: 6 or variants thereof;
e. GA comprising the amino acid sequence of SEQ ID NO: 7 or variants thereof;
f. IN comprising the amino acid sequence of SEQ ID NO: 8 or variants thereof;
g. IV comprising the amino acid sequence of SEQ ID NO: 9 or variants thereof;
h. KP comprising the amino acid sequence of SEQ ID NO: 10 or variants thereof;
i. LZ comprising the amino acid sequence of SEQ ID NO: 11 or variants thereof; and
j. SA comprising the amino acid sequence of SEQ ID NO: 12 or variants thereof;
and wherein the signal peptides enable the extracellular secretion of polypeptide comprising the amino acid sequence of SEQ ID NO: 1.

3. A nucleic acid comprising the nucleotide sequence of SEQ ID NO: 2.

4. A nucleic acid encoding the polypeptide as claimed in claim 1.

5. The nucleic acid as claimed in claim 4, wherein the nucleic acid is selected from a group comprising SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and variants thereof.

6. An expression vector comprising a nucleic acid as claimed in claim 3 operably linked to a promoter.

7. The expression vector as claimed in claim 6, wherein the promoter for β-fructofuranosidase gene is selected from group comprising AOX1, ADH3, DAS, FLD1, LRA3, THI11, GAP, YPT1, TEF1, GCw14 and PGK1.

8. The expression vector as claimed in claim 6, wherein vector is selected from a group comprising pPICZαA, pPICZαB, pPICZαC, pGAPZαA, pGAPZαB, pGAPZαC, pPIC3, pPIC3.5, pPIC3.5K, PAO815, pPIC9, pPIC9K, IL-D2, pHIL-S1 and expression vectors configured for secretory or intracellular expression of β-fructofuranosidase from Aspergillus niger as set forth in SEQ ID NO: 1.

9. A recombinant Pichia pastoris host cell comprising an expression vector as claimed in claim 6.

10. The recombinant Pichia pastoris host cell as claimed in claim 9, wherein the host cell is selected from a group comprising Pichia pastoris Mut+, Mut S, Mut−, Pichia pastoris KM71H, Pichia pastoris KM71, Pichia pastoris SMD1168H, Pichia pastoris SMD1168, Pichia pastoris X33, Pichia pastoris GS115 or any other Pichia pastoris host strain.

11. A method of producing a recombinant Pichia pastoris host cell according to claim 9 capable of expressing β-fructofuranosidase of Aspergillus niger as set forth in SEQ ID NO: 1, said process comprising the steps of:
a. synthesizing a modified nucleic acid encoding β-fructofuranosidase from Aspergillus niger as set forth in SEQ ID NO: 1 or variants thereof;
b. constructing a vector harboring the modified nucleic acid; and
c. transforming a Pichia pastoris host cell with the vector of step (b) to obtain a recombinant Pichia pastoris host cell.

12. A process for expressing β-fructofuranosidase of Aspergillus niger as set forth in SEQ ID NO: 1 according to claim 1 at high levels, comprising:
a. culturing recombinant Pichia pastoris host cells capable of expressing β-fructofuranosidase of Aspergillus niger as set forth in SEQ ID NO: 1 in a suitable fermentation medium to obtain a fermentation broth;
b. harvesting supernatant from the fermentation broth, wherein the supernatant contains recombinant β-fructofuranosidase; and
c. purifying recombinant β-fructofuranosidase.

13. The process as claimed in claim 12, wherein the fermentation medium is Basal Salt Media.

14. The process as claimed in claim 12, wherein the pH of the fermentation broth is maintained in the range from 4.0 to 7.5.

15. The process as claimed in claim 12, wherein the temperature of the fermentation broth in maintained in the range from 15° C. to 45° C.

16. The modified polypeptide as claimed in claim 1 or a fragment thereof for use in production of fructooligosaccharides.

17. The modified polypeptide or a fragment thereof for use in production of fructooligosaccharides as claimed in claim 16, wherein the fragment is selected from a group comprising SEQ ID NO: 24, SEQ ID NO: 25, SEQ ID NO: 26 and SEQ ID NO: 27.


SEQUENCE LISTING (27 sequences)

SEQ ID NO: 1 - protein, 654 aa, Aspergillus niger
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 2 - DNA, 1965 bp, Artificial Sequence
  Modified nucleic acid sequence of the gene encoding beta-fructofuranosidase
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 3 - protein, 85 aa, Artificial Sequence
  Modified Alpha-factor of S. cerevisiae (FAK)
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 4 - protein, 89 aa, Artificial Sequence
  Modified Alpha-factor full of S. cerevisiae (FAKS)
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 5 - protein, 23 aa, Artificial Sequence
  Modified Alpha factor_T of S. cerevisiae (AT)
MRFPSIFTAVLFAASSALALEKR

SEQ ID NO: 6 - protein, 24 aa, Artificial Sequence
  Modified Alpha-amylase of Aspergillus niger (AA)
MVAWWSLFLYGLQVAAPALALEKR

SEQ ID NO: 7 - protein, 22 aa, Artificial Sequence
  Modified Glucoamylase of Aspergillus awamori (GA)
MSFRSLLALSGLVCSGLALEKR

SEQ ID NO: 8 - protein, 20 aa, Artificial Sequence
  Modified Inulinase of Kluyveromyces maxianus (IN)
MKLAYSLLLPLAGVSALEKR

SEQ ID NO: 9 - protein, 23 aa, Artificial Sequence
  Modified Invertase of S. cerevisiae (IV)
MLLQAFLFLLAGFAAKISALEKR

SEQ ID NO: 10 - protein, 30 aa, Artificial Sequence
  Modified Killer protein of S. cerevisiae (KP)
MTKPTQVLVRSVSILFFITLLHLVVALEKR

SEQ ID NO: 11 - protein, 30 aa, Artificial Sequence
  Modified Lysozyme of Gallus gallus (LZ)
MLGKNDPMCLVLVLLGLTALLGICQGLEKR

SEQ ID NO: 12 - protein, 22 aa, Artificial Sequence
  Modified Serum albumin of Homo sapiens (SA)
MKWVTFISLLFLFSSAYSLEKR

SEQ ID NO: 13 - DNA, 2220 bp, Artificial Sequence
  Alpha-factor (FAK) of S. cerevisiae fused to modified nucleic acid of beta-fructofuranosidase gene
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 14 - DNA, 2232 bp, Artificial Sequence
  Alpha-factor full (FAKS) of S. cerevisiae fused to modified nucleic acid of beta-fructofuranosidase gene
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaagagagaggctgaagctatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 15 - DNA, 2034 bp, Artificial Sequence
  Alpha-factor_T (AT) of S. cerevisiae fused to modified nucleic acid of beta-fructofuranosidase gene
atgagatttccttcaatttttactgctgttttattcgcagcatcctccgcattagctctcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 16 - DNA, 2037 bp, Artificial Sequence
  Alpha-amylase (AA) of Aspergillus niger fused to modified nucleic acid of beta-fructofuranosidase gene
atggttgcttggtggagtcttttcctatacggtctacaggtggcagctccagcccttgccctcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 17 - DNA, 2031 bp, Artificial Sequence
  Glucoamylase (GA) of Aspergillus awamori fused to modified nucleic acid of beta-fructofuranosidase gene
atgtctttccgatctcttttagccctatctggacttgtttgttcaggtttggctctcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 18 - DNA, 2025 bp, Artificial Sequence
  Inulinase (IN) of Kluyveromyces maxianus fused to modified nucleic acid of beta-fructofuranosidase gene
atgaagttggcttattctcttcttcttcctctggccggagtgtctgccctcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 19 - DNA, 2034 bp, Artificial Sequence
  Invertase (IV) of S.cerevisiae fused to modified nucleic acid of beta-fructofuranosidase gene
atgcttttgcaggctttcctgttcttgctggccggattcgctgctaaaatttccgctctcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 20 - DNA, 2055 bp, Artificial Sequence
  Killer protein (KP) of S.cerevisiae fused to modified nucleic acid of beta-fructofuranosidase gene
atgaccaaaccaactcaagttttggtgaggtctgtgtcaatcctgttcttcattactttactgcaccttgtagtcgcactcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 21 - DNA, 2055 bp, Artificial Sequence
  Lysozyme (LZ) of Gallus gallus fused to modified nucleic acid of beta-fructofuranosidase gene
atgctaggcaaaaatgaccctatgtgtttggttctggttttgcttggtttaaccgctttacttggtatctgtcaaggtctcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 22 - DNA, 2031 bp, Artificial Sequence
  Serum albumin (SA) of Homo sapiens fused to modified nucleic acid of beta-fructofuranosidase gene
atgaagtgggtaacatttatttccctactgtttcttttttcttcagcttactctctcgagaagagaatgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 23 - DNA, 1965 bp, Aspergillus niger
atgaagctcaccactaccaccctggcgctcgccaccggcgcagcagcagcagaagcctcataccacctggacaccacggccccgccgccgaccaacctcagcaccctccccaacaacaccctcttccacgtgtggcggccgcgcgcgcacatcctgcccgccgagggccagatcggcgacccctgcgcgcactacaccgacccatccaccggcctcttccacgtggggttcctgcacgacggggacggcatcgcgggcgccaccacggccaacctggccacctacaccgatacctccgataacgggagcttcctgatccagccgggcgggaagaacgaccccgtcgccgtgttcgacggcgccgtcatccccgtcggcgtcaacaacacccccaccttactctacacctccgtctccttcctgcccatccactggtccatcccctacacccgcggcagcgagacgcagtcgttggccgtcgcgcgcgacggcggccgccgcttcgacaagctcgaccagggccccgtcatcgccgaccaccccttcgccgtcgacgtcaccgccttccgcgatccgtttgtcttccgcagtgccaagttggatgtgctgctgtcgttggatgaggaggtggcgcggaatgagacggccgtgcagcaggccgtcgatggctggaccgagaagaacgccccctggtatgtcgcggtctctggcggggtgcacggcgtcgggcccgcgcagttcctctaccgccagaacggcgggaacgcttccgagttccagtactgggagtacctcggggagtggtggcaggaggcgaccaactccagctggggcgacgagggcacctgggccgggcgctgggggttcaacttcgagacggggaatgtgctcttcctcaccgaggagggccatgacccccagacgggcgaggtgttcgtcaccctcggcacggaggggtctggcctgccaatcgtgccgcaggtctccagtatccacgatatgctgtgggcggcgggtgaggtcggggtgggcagtgagcaggagggtgccaaggtcgagttctccccctccatggccgggtttctggactgggggttcagcgcctacgctgcggcgggcaaggtgctgccggccagctcggcggtgtcgaagaccagcggcgtggaggtggatcggtatgtctcgttcgtctggttgacgggcgaccagtacgagcaggcggacgggttccccacggcccagcaggggtggacggggtcgctgctgctgccgcgcgagctgaaggtgcagacggtggagaacgtcgtcgacaacgagctggtgcgcgaggagggcgtgtcgtgggtggtgggggagtcggacaaccagacggccaggctgcgcacgctggggatcacgatcgcccgggagaccaaggcggccctgctggccaacggctcggtgaccgcggaggaggaccgcacgctgcagacggcggccgtcgtgccgttcgcgcaatcgccgagctccaagttcttcgtgctgacggcccagctggagttccccgcgagcgcgcgctcgtccccgctccagtccgggttcgaaatcctggcgtcggagctggagcgcacggccatctactaccagttcagcaacgagtcgctggtcgtcgaccgcagccagactagtgcggcggcgcccacgaaccccgggctggatagctttactgagtccggcaagttgcggttgttcgacgtgatcgagaacggccaggagcaggtcgagacgttggatctcactgtcgtcgtggataacgcggttgtcgaggtgtatgccaacgggcgctttgcgttgagcacctgggcgagatcgtggtacgacaactccacccagatccgcttcttccacaacggcgagggcgaggtgcagttcaggaatgtctccgtgtcggaggggctctataacgcctggccggagagaaattga

SEQ ID NO: 24 - protein, 6 aa, Artificial Sequence
  Conserved bioactive fragment of beta- fructofuranosidase of Aspergillus niger (Position 57-62)
QIGDPC

SEQ ID NO: 25 - protein, 14 aa, Artificial Sequence
  Conserved bioactive fragment of beta- fructofuranosidase of Aspergillus niger (Position 119-132)
DGAVIPVGVNNTPT

SEQ ID NO: 26 - protein, 10 aa, Artificial Sequence
  Conserved bioactive fragment of beta- fructofuranosidase of Aspergillus niger (Position 320-330)
SGLPIVPQVS

SEQ ID NO: 27 - protein, 16 aa, Artificial Sequence
  Conserved bioactive fragment of beta- fructofuranosidase of Aspergillus niger (Position 401-416)
GDQYEQADGFPTAQQG